(19) United States
(12) Patent Application Publication
(10) Pub. No.: US 2020/0384189 A1
(43) Pub. Date: Dec. 10, 2020

(54) A DUAL ACTIVE VALVE FLUID PRESSURE OPERATED POSITIVE DISPLACEMENT PUMP

(71) Applicant: SERENNO MEDICAL, Yokneam llit (IL)

(72) Inventors: Tomer LARK, Herzeliya (IL); Noam HADAS, Tel Aviv (IL)

(21) Appl. No.: 16/767,692

(22) PCT Filed: Nov. 29, 2018
(86) PCT No.: PCT/IL2018/051311

§ 371 Date: May 28, 2020

Related U.S. Application Data
(60) Provisional application No. 62/591,803, filed on Nov. 29, 2017.

(51) Int. Cl. A61M 5/142; F04B 43/067

(52) U.S. Cl. CPC A61M 5/14224; F04B 43/073; F04B 43/067

(57) ABSTRACT

A dual active valve positive displacement pump comprising: A housing holding the pump's components. A piston with an internal cavity divided into two fluidly-isolated volumes by a freely-moving diaphragm, one of the two volumes being fluidly connected with a volume between piston and housing which contains driver pressure from a pressure source. The piston is reciprocally movable inside the housing under positive or negative driver pressure. An active inlet valve operable by driver pressure actuates when the driver pressure is more than the maximum pressure at the pump inlet port. An active outlet valve operable by driver pressure actuates when the driver pressure is less than the minimum pressure at the pump outlet port. The diaphragm separates pumped fluid from operational fluid used to move the diaphragm inside the piston cavity and transmits pressure at the inlet port when the inlet valve is open, and at the outlet port when the outlet valve is open.

### FIELD OF THE INVENTION

The present invention generally pertains to a device for pumping fluid that is accurate and reliable and provides a precise flow rate and a constant stroke volume.

### BACKGROUND OF THE INVENTION

In the art of pumping fluids it is frequently desirable to provide precise flow rates or stroke volumes. A positive displacement pump can be used for such applications, but normally the valves that feed the pump cavity are passive, operable only by the difference in pressures between the inlet or outlet ports and the pressure inside the pump cavity. Such valves are usually leaf valves, and since their opening and closing is operable by unknown pressure differences, their timing and speed of operation are unknown and variable—leading to uncontrolled changes in the pump throughput or stroke volume.

More sophisticated positive displacement pumps make use of active, driven valves to insure constant and accurate opening and closing timing and speeds. These active valves may be operable through electric, magnetic or hydraulic actuators, separate from the main pumping actuator, which are added to the basic pump design making it more complex and less reliable.

Examples of bellows, membrane or positive displacement pump are given below.

A patent issued to Peer M. Portner et al (U.S. Pat. No. 4,265,241) discloses a bellows pump consisting of a piston bellows which is actuated by a solenoid controlled armature. Movement of the piston bellows tends to increase or decrease the volume of the pumping chamber. When the volume of the pump chamber is a maximum, the pumped fluid is forced from a reservoir, which is maintained at positive pressure, through an input passive check valve into the pump chamber. When the bellows piston is actuated, the pump chamber is at a minimum volume and fluid is forced out of the chamber through another output passive check valve.

U.S. Pat. No. 4,360,019 to Portner et al describes a positive displacement pump which uses an elastomeric diaphragm which is driven by a solenoid via a plunger. Movement of a diaphragm varies the volume in the pump cavity which causes fluid to flow into the cavity via a passive spring-loaded inlet valve or to flow out of the cavity via a different passive spring-loaded outlet valve.

U.S. Pat. No. 4,152,098, issued to Norman F. Moody et al discloses a pump having a diaphragm which forms the inlet valve, outlet valve, and is the moveable member which varies the volume in the pumping chamber. A solenoid actuated ball is driven in contact with the diaphragm to vary the volume in the pumping chamber. Although the diaphragm remains in conformity with the ball, differential pressure across the input valve will cause the stroke volume of this prior art pump to vary.

Several of the above-cited references teach the use of a compliant diaphragm or bellows which results in variations in pump stroke volume with changes in the reservoir pressure or in ambient pressure conditions. Additionally, all of the above-cited references teach the use of passive inlet and outlet valves. Since the flow rate through the valve depends upon differential pressure across the valve, the flow rate through the inlet valve and therefore the stroke volume is dependent upon ambient pressure and reservoir pressure. Therefore, the prior art research and experimentation with various types of pumps has not provided a positive displacement pump which has an accurate and constant stroke volume.

It is therefore a long felt need to provide a pump without uncontrolled changes in the pump throughput or stroke volume where there is no contact between operating fluid and pumped fluid, no pressure sensors are in contact with the pumped fluid, which measures input and output pressures but does not need a pressure sensor on either the input or output line.

### SUMMARY OF THE INVENTION

It is an object of the present invention to disclose a system for pumping fluid that is accurate and reliable and provides a precise flow rate and a constant stroke volume.

It is another object of the present invention to disclose a positive displacement pump comprising:
a housing having at least two pumping ports for flowing a pumpable fluid into and out of the pump and at least one control port for flowing operating pressure into and out of the pump;
at least one cavity inside the housing, divided into a first volume and a second volume by a freely movable divider, said first volume fluidly isolated from said second volume, said second volume fluidly connectable to said at least two pumping ports, said first volume fluidly connected to said at least one control port; said movable divider movable by means of said operating pressure; said second volume reversibly enlargeable by movement of said movable divider; and
at least two valves configured to control flow through said at least two pumping ports, fluid flow through all said at least two pumping ports controllable by at least one of said at least two valves, a first at least one of said at least two valves in fluid connection with a first at least one of said at least two pumping ports, a second at least one of said at least two valves in fluid connection with a second at least one of said at least two pumping ports, control of at least one of said at least two valves being independent of control of at least one other of said at least two valves;
wherein enlargement of said second volume flows said pumpable fluid into said pump and reversing enlargement of said second volume flows said pumpable fluid out of said pump.

It is another object of the present invention to disclose the pump as described above, wherein control of at least one of said at least two valves is independent of control of said operating pressure.

It is another object of the present invention to disclose the pump as described above, additionally comprising at least one vent port in fluid communication with said second volume.

It is another object of the present invention to disclose the pump as described above, wherein a pump stroke comprises one said enlargement of said second volume and one said reversal of said enlargement.

It is another object of the present invention to disclose the pump as described above, wherein a predetermined volume of fluid is transferred from said at least one first port to said at least one second port during each said pump stroke.

It is another object of the present invention to disclose the pump as described above, wherein the divider separating the cavity into said first volume and said second volume is a flexible plastic film, with a thickness in a range of 0.01 mm-0.5 mm.

It is another object of the present invention to disclose the pump as described above, wherein said first volume is fluidly isolated from said second volume by said divider.

It is another object of the present invention to disclose the pump as described above, wherein at least one of said at least two valves is a pinch valve.

It is another object of the present invention to disclose the pump as described above, wherein said pinch valve is selected from a group consisting of a pneumatic-controlled valve, a hydraulic-controlled valve, a motor-driven valve, and a solenoid operated valve.

It is another object of the present invention to disclose a positive displacement pump comprising:
a housing having at least three fluid ports, at least one inlet port configured to allow flow of a pumped fluid into the pump, at least one outlet port configured to allow flow of said pumped fluid out of the pump, and at least one driver pressure port configured to allow operating pressure into and out of the pump;
at least one main piston movable within a space defined by at least one first interior wall of the housing between a first stable position and a second stable position under said operating pressure, said operating pressure acting in a space between the main piston and at least one second interior wall of the housing;
at least one cavity coupled to or formed inside the main piston, said cavity divided into a first volume and a second volume by a freely moving divider;
at least one first valve, operable by motion of said main piston, configured to provide fluid connection between said at least one inlet port and said first volume when the main piston in said first stable position; and
at least one second valve, operable by the motion of said main piston, configured to provide fluid connection between said at least one outlet port and said first volume when the main piston in said second stable position;
wherein said pump is operable by a single pressure, said operating pressure, pumping of fluid between the inlet port and the outlet port being controlled by said operating pressure, and fluid pressure at said inlet port and said outlet port determinable from measurement of said operating pressure.

It is another object of the present invention to disclose the pump as described above, wherein the divider separating the cavity into said first volume and said second volume is selected from a group consisting of a diaphragm and a second piston.

It is another object of the present invention to disclose the pump as described above, wherein said diaphragm comprises a flexible plastic film with a thickness in a range of 0.01 mm-0.5 mm.

It is another object of the present invention to disclose the pump as described above, wherein the pressure at the inlet port is determinable from the pressure at the driver pressure port, during a time in which a change of driver fluid volume in the space between the main piston and at least one second interior wall of the housing does not result in a pressure change in said space between the main piston and said at least one second interior wall of the housing, and the main piston in the said first position.

It is another object of the present invention to disclose the pump as described above, wherein the pressure at the outlet port is determinable from the pressure at the driver pressure port, during a time in which a change of driver fluid volume in the space between the main piston and said at least one second interior wall of the housing does not result in a pressure change in said space between the main piston and said at least one second interior wall of the housing, and the main piston in the said second position.

It is another object of the present invention to disclose the pump as described above, wherein the driver pump inverts its direction of operation after detection of a sudden drop in absolute pressure in the space between the main piston and said at least one second interior wall of the housing, said sudden drop on absolute pressure indicating that the main piston has moved from one stable position to the other stable position.

It is another object of the present invention to disclose the pump as described above, wherein the pressure at the outlet or the inlet port is calculable by a process comprising the steps of:
measuring several data points on the curve representing the relationship between a change in volume of driver fluid and the pressure in the space between the main piston and said at least one second interior wall of the housing, said pressure being equal to pressure at the driver pump port;
calculating the parameters of two straight lines for the process of increasing the pressure from minimum value to maximum value, and two straight lines for the process of decreasing the pressure from the maximum value to the minimum value, having a formula as P₁=a₁(ΔV)+b₁ and P₂=a₂(ΔV)+b₂. One line is before the diaphragm moves from one stable position to another stable position, thereby either increasing or decreasing volume of the space between the main piston and said at least one second interior wall of the housing, and a second line after the diaphragm moves from one position to another position;
calculating the P₁=P₂ where a₁(ΔV)+b₁=a₂(ΔV)+b₂ where ΔV is equal to the volume of the cavity inside the main piston; and
from the pressure at the driver pressure port, when a change of driver fluid volume in the space between the main piston and said at least one second interior wall of the housing does not result in a pressure change in said space between the main piston and said at least one second interior wall of the housing, and the main piston is in the said second position.

It is another object of the present invention to disclose the pump as described above, wherein a volume in the cavity available to the pumpable fluid is measurable as a change in volume of the driver fluid, said change in volume of the driver fluid not inducing a change in pressure in the space between the main piston and said at least one second interior wall of the housing.

It is another object of the present invention to disclose the pump as described above, wherein the driver pump reverses its direction of operation from increasing the pressure to decreasing the pressure and vice versa, at such time as is measured a sudden drop in the absolute value of the pressure in the space between the main piston and said at least one second interior wall of the housing said sudden drop in pressure indicating that the main piston has moved from one stable position to the other stable position.

It is another object of the present invention to disclose the pump as described above, wherein a leak in a fluid connection between a driver pressure pump and the driver pressure port is detectable from a continuation of the change in measured pressure towards zero pressure after the driver pressure pump, having a non-zero driver pressure, is stopped.

It is another object of the present invention to disclose the pump as described above, wherein a malfunction condition of the main piston is detectable from a smaller-than-normal drop in absolute pressure when the main piston is moving at either maximal or minimal control pressure, said malfunction being failure of said main piston to move fully from one stable position to the other stable position.

It is another object of the present invention to disclose the pump as described above, wherein at least one volume of pumped fluid smaller than a usable volume of the cavity is releasable by the pump, by operating the driver pressure pump when the control pressure is equal to the outlet port pressure, and injecting a predetermined volume of driver fluid smaller than a usable volume of the cavity.

It is another object of the present invention to disclose the pump as described above, wherein the diaphragm dividing the cavity lies in a plane that is not perpendicular to the axis of motion of the moving part.

It is another object of the present invention to disclose the pump as described above, wherein each of said inlet port and said outlet port comprise a pair of holes, said pair of holes being a hole in said main piston matched to a hole in a portion of said at least one first wall of said housing, and said pair of holes have a dimension along an axis of motion of the main piston in a range between 1.3 and 5 times smaller than a dimension along an axis perpendicular to the axis of motion of the main piston.

It is another object of the present invention to disclose a method of operating a positive displacement pump comprising steps of:
providing a positive displacement pump comprising:
a housing having at least two pumping ports for flowing a pumpable fluid into and out of the pump and at least one control port for flowing operating pressure into and out of the pump;
at least one cavity inside the housing, divided into a first volume and a second volume by a freely movable divider, said first volume fluidly isolated from said second volume, said second volume fluidly connectable to said at least two pumping ports, said first volume fluidly connected to said at least one control port; said movable divider movable by means of said operating pressure; said second volume reversibly enlargeable by movement of said movable divider; and
at least two valves configured to control flow through said at least two pumping ports, fluid flow through all said at least two pumping ports controllable by at least one of said at least two valves, a first at least one of said at least two valves in fluid connection with a first at least one of said at least two pumping ports, a second at least one of said at least two valves in fluid connection with a second at least one of said at least two pumping ports, control of at least one of said at least two valves being independent of control of at least one other of said at least two valves;
connecting at least one of said at least two pumping ports to a source of fluid; and
executing at least one pump stroke, said pump stroke comprising:
   opening said first at least one of said at least two valves and closing said second at least one of said at least two valves and decreasing said operating pressure, thereby enlarging said second volume and flowing said pumpable fluid into said second volume; and
   at such time as said second volume is fully enlarged, closing said first at least one of said at least two valves and opening said second at least one of said at least two valves and increasing said operating pressure, thereby reversing enlargement of said second volume and flowing said pumpable fluid out of said pump.

It is another object of the present invention to disclose the method as described above, additionally comprising a step of connecting at least one other of said at least two pumping ports to a reservoir or drain for disposing of said fluid.

It is another object of the present invention to disclose the method as described above, additionally comprising a step of controlling at least one of said at least two valves independently of control of said operating pressure.

It is another object of the present invention to disclose the method as described above, additionally comprising a step of providing at least one vent port in fluid communication with said second volume.

It is another object of the present invention to disclose the method as described above, additionally comprising a step of executing a pump stroke comprising one said enlargement of said second volume and one said reversal of said enlargement.

It is another object of the present invention to disclose the method as described above, additionally comprising a step of transferring a predetermined volume of fluid from said at least one first port to said at least one second port during each said pump stroke.

It is another object of the present invention to disclose the method as described above, additionally comprising a step of providing the divider separating the cavity into said first volume and said second volume as a flexible plastic film with a thickness in a range of 0.01 mm-0.5 mm.

It is another object of the present invention to disclose the method as described above, additionally comprising a step of fluidly isolating said first volume from said second volume by said divider.

It is another object of the present invention to disclose the method as described above, additionally comprising a step of providing at least one of said at least two valves as a pinch valve.

It is another object of the present invention to disclose the method as described above, additionally comprising a step of selecting said pinch valve from a group consisting of a pneumatic-controlled valve, a hydraulic-controlled valve, a motor-driven valve, and a solenoid operated valve.

It is another object of the present invention to disclose a method of operating a positive displacement pump comprising steps of:
providing a positive displacement pump comprising:
   a housing having at least three fluid ports, at least one inlet port configured to allow flow of a pumped fluid into the pump, at least one outlet port configured to allow flow of said pumped fluid out of the pump, and at least one driver pressure port configured to allow operating pressure into and out of the pump;
   at least one main piston movable within a space defined by at least one first interior wall of the housing between a first stable position and a second stable position under said operating pressure, said operating pressure acting in a space between the main piston and at least one second interior wall of the housing;
   at least one cavity coupled to or formed inside the main piston, said cavity divided into a first volume and a second volume by a freely moving divider;
   at least one first valve, operable by motion of said main piston, configured to provide fluid connection between said at least one inlet port and said first volume when the main piston in said first stable position; and
   at least one second valve, operable by the motion of said main piston, configured to provide fluid connection between said at least one outlet port and said first volume when the main piston in said second stable position;
connecting said at least one inlet port to a source of fluid; and
executing at least one pump stroke, said pump stroke comprising:
   with said main piston in a down position and said divider in a lower position, the inlet valve being open, and said operating pressure greater than an inlet pressure, lowering the operating pressure;
   continuing to lower said operating pressure until said operating pressure is lower than said inlet pressure, said operating pressure being lower than said inlet pressure moving said divider to an upper position, thus enlarging said first volume and flowing said pumpable fluid into said first volume;
   continuing to lower said operating pressure until said main piston moves from said down position to an up position, said moving of said main piston closing said inlet valve and opening said outlet valve;
   raising said operating pressure until said operating pressure is greater than said outlet pressure, said operating pressure being greater than said outlet pressure moving said divider to a lower position, thus reducing said first volume and flowing said pumpable fluid out of said first volume; and
   continuing to raise said operating pressure until said main piston moves from said up position to said down position, said moving of said main piston closing said outlet valve and opening said inlet valve;
wherein said pump is operable by a single pressure, said operating pressure, pumping of fluid between the inlet port and the outlet port being controlled by said operating pressure, and fluid pressure at said inlet port and said outlet port determinable from measurement of said operating pressure.

It is another object of the present invention to disclose the method as described above, additionally comprising a step of selecting the divider separating the cavity into said first volume and said second volume from a group consisting of a diaphragm and a second piston.

It is another object of the present invention to disclose the method as described above, additionally comprising a step of said diaphragm comprising a flexible plastic film with a thickness in a range of 0.01 mm-0.5 mm.

It is another object of the present invention to disclose the method as described above, additionally comprising a step of determining the pressure at the inlet port from the pressure at the driver pressure port, during a time in which a change of driver fluid volume in the space between the main piston and at least one second interior wall of the housing does not result in a pressure change in said space between the main piston and said at least one second interior wall of the housing, and the main piston in the said first position.

It is another object of the present invention to disclose the method as described above, additionally comprising a step of determining the pressure at the outlet port from the pressure at the driver pressure port, during a time in which a change of driver fluid volume in the space between the main piston and said at least one second interior wall of the housing does not result in a pressure change in said space between the main piston and said at least one second interior wall of the housing, and the main piston in the said second position.

It is another object of the present invention to disclose the method as described above, additionally comprising a step of the driver pump inverting its direction of operation after detection of a sudden drop in absolute pressure in the space between the main piston and said at least one second interior wall of the housing, said sudden drop on absolute pressure indicating that the main piston has moved from one stable position to the other stable position.

It is another object of the present invention to disclose the method as described above, additionally comprising a step of calculating the pressure at the outlet or the inlet port by a process comprising the steps of:
measuring several data points on the curve representing the relationship between a change in volume of driver fluid and the pressure in the space between the main piston and said at least one second interior wall of the housing, said pressure being equal to pressure at the driver pump port;
calculating the parameters of two straight lines for the process of increasing the pressure from minimum value to maximum value, and two straight lines for the process of decreasing the pressure from the maximum value to the minimum value, having a formula as P₁=a₁(ΔV)+b₁ and P₂=a₂(ΔV)+b₂. One line is before the diaphragm moves from one stable position to another stable position, thereby either increasing or decreasing volume of the space between the main piston and said at least one second interior wall of the housing, and a second line after the diaphragm moves from one position to another position;
calculating the P₁=P₂ where a₁(ΔV)+b₁=a₂(ΔV)+b₂ where ΔV is equal to the volume of the cavity inside the main piston; and
from the pressure at the driver pressure port, when a change of driver fluid volume in the space between the main piston and said at least one second interior wall of the housing does not result in a pressure change in said space between the main piston and said at least one second interior wall of the housing, and the main piston is in the said second position.

It is another object of the present invention to disclose the method as described above, additionally comprising a step of measuring a volume in the cavity available to the pumpable fluid as a change in volume of the driver fluid, said change in volume of the driver fluid not inducing a change in pressure in the space between the main piston and said at least one second interior wall of the housing.

It is another object of the present invention to disclose the method as described above, additionally comprising a step of the driver pump reversing its direction of operation from increasing the pressure to decreasing the pressure and vice versa, at such time as is measured a sudden drop in the absolute value of the pressure in the space between the main piston and said at least one second interior wall of the housing said sudden drop in pressure indicating that the main piston has moved from one stable position to the other stable position.

It is another object of the present invention to disclose the method as described above, additionally comprising a step of detecting a leak in a fluid connection between a driver pressure pump and the driver pressure port is detectable from a continuation of the change in measured pressure towards zero pressure after the driver pressure pump, having a non-zero driver pressure, is stopped.

It is another object of the present invention to disclose the method as described above, additionally comprising a step of detecting a malfunction condition of the main piston from a smaller-than-normal drop in absolute pressure when the main piston is moving at either maximal or minimal control pressure, said malfunction being failure of said main piston to move fully from one stable position to the other stable position.

It is another object of the present invention to disclose the method as described above, additionally comprising a step of the pump releasing at least one volume of pumped fluid smaller than a usable volume of the cavity, by operating the driver pressure pump when the control pressure is equal to the outlet port pressure, and injecting a predetermined volume of driver fluid smaller than a usable volume of the cavity.

It is another object of the present invention to disclose the method as described above, additionally comprising a step of providing the diaphragm dividing the cavity lying in a plane that is not perpendicular to the axis of motion of the moving part.

It is another object of the present invention to disclose the method as described above, additionally comprising a step of, each of said inlet port and said outlet port comprising a pair of holes, said pair of holes being a hole in said main piston matched to a hole in a portion of said at least one first wall of said housing, providing said pair of holes having a dimension along an axis of motion of the main piston in a range between 1.3 and 5 times smaller than a dimension along an axis perpendicular to the axis of motion of the main piston.

It is another object of the present invention to disclose the method as described above, additionally comprising a step of connecting said outlet port to a reservoir or drain for disposing of said fluid.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1 illustrates a detailed view of an embodiment of the pump;

FIG. 2A-H illustrates the eight phases in the operation of the pump in series in a complete pumping cycle;

FIG. 3 illustrates the driver pressure vs. driver volume change though a typical pumping cycle, and the relative pump state in each phase of the cycle;

FIG. 4 illustrates an embodiment of an algorithm to detect inlet or outlet pressure from the change in slope in the volume/pressure pumping loop;

FIG. 5 illustrates a second embodiment of the pump; and

FIG. 6 illustrates the driver pressure vs. driver volume change though a typical pumping cycle, and the relative pump state in each phase of the cycle.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for pumping fluid that provides a precise flow rate and a constant stroke volume and is accurate and reliable

The term ‘cavity divider’ hereinafter refers to the diaphragm (membrane), piston or other movable separator inside the cavity in the movable element. The cavity divider subdivides the cavity into two fluidly-isolated sub-volumes.

The term ‘main piston’ hereinafter refers to a movable part of the pump. The main piston comprises an opening connectable to an inlet port, an opening to an outlet port, an opening fluidly connected to a pressure source and a cavity.

The term ‘pump stroke’ hereinafter refers to a single cycle of operation of a pump, each pump stroke configured to transfer a fixed quantity of a pumpable fluid from a first port of the pump, acting as an inlet port, to a second port of the pump, acting as an outlet port.

The present invention is a positive displacement pump which can deliver precise and repeatable stroke volumes. Additionally, the present invention teaches a positive displacement pump which is operable through a single pressure lead connected to a positive and negative pressurized fluid source. Additionally, the present invention teaches the design of a positive displacement pump which facilitates the measurement of pressures at the inlet and at the outlet ports through the same lead used for providing the pressures that drive the pump. The pump also has a stroke volume which is constant; the stroke volume is independent of ambient pressure and reservoir pressure over a considerable range.

The pump of the present invention comprises a rigid housing containing a moveable part, the main piston. Inside the main piston is an internal cavity of a known and fixed volume. The cavity is subdivided into two fluidly-isolated sub-volumes by a movable cavity divider. In some embodiments, the cavity divider is a very flexible diaphragm or membrane embedded in the cavity, with the diaphragm dividing the volume of the cavity into the two fluidly-isolated sub-volumes. In some embodiments, a freely moving piston having sealing elements can divide the volume of the cavity into the two fluidly-isolated sub-volumes. The two sub-volumes are hereby denoted the upper portion of the cavity and the lower portion of the cavity. The upper portion is in fluid connection with the space between the moving part and the housing and is therefore exposed to the driver pressure.

The cavity divider is reversibly moveable from a first position where it is resting fully against the upper wall of the pump cavity, so that the lower portion comprises the entire usable volume of the cavity, to a second position where the cavity divider is resting fully on the lower wall of the pump cavity, so that the upper portion comprises the entire usable volume of the cavity. Any state between these two positions is an intermediate position. Intermediate positions are not stable during normal operation. The cavity divider can be driven from the first position to the second and vice versa by a source of fluid pressure, this source of fluid pressure being fluidly connected to the upper portion of the pump cavity. The cavity divider can freely move up and down in the cavity with minimal losses due to bending of the diaphragm or friction with the cavity walls while maintaining fluid isolation between the upper and lower portions of the cavity. This means that, whenever the pressure in the upper and lower portions of the cavity are equal, the cavity divider will experience zero net force and will be free to move under the slightest imbalance in pressure between the two portions.

When the fluid pressure supplied by the driver source is higher than the pressure at the outlet port and the outlet valve is open, the cavity divider is driven down from the first position to the second position, minimizing the volume of the lower portion of the pump cavity. When the fluid pressure supplied by the driver source is lower than the pressure at the inlet port when the inlet valve is open, the cavity divider is driven up from the second position to the first position, maximizing the volume of the lower portion of the pump cavity.

The lower portion of the pump cavity is fluidly connectable to two valves, one which can connect the lower portion of the pump cavity to the inlet port and one which can connect the lower portion of the pump cavity to the outlet port. These valves have two stable positions, OPEN and CLOSED. When the moveable part is in the down position, the first valve is open, fluidly connecting the lower portion of the pump cavity to the inlet port which is connected to a source for the pumped fluid. In the up position of the moveable part, fluid connection between the inlet port and the first valve is broken, not allowing any fluids to pass through. At the same time, a second valve opens a fluid connection between the pump cavity and the outlet port of the pump. These valves are operable through a force generated by a driver fluid pressure provided to a driver port, fluidly connected to some mechanism which converts this pressure to the force necessary to implement the mechanical motion which moves the valve element from one stable position to the other and back. This pressure source can be a powered pressure generating device such as a piston pump, a peristaltic pump or some other pump.

Both pressure controlled valves are fluidly connected to the same source of fluid pressure already described as driving the cavity divider inside the pump cavity between its first and second positions.

The positive and negative pressures at which the inlet and outlet valves are switched from the up position to the down position and vice versa are much higher than any pressure which is expected to appear in normal operation either at the inlet port or at the outlet port for the pumped fluid.

In operation, a pumping cycle begins with the driving pressure source delivering a first pressure level which is sufficient to A. Move the main piston to the down position, and B. move the cavity divider to the second position where the volume of the lower portion of the pump cavity is zero, because the cavity divider is resting against the lower wall of the cavity.

In this first state, the lower portion of the cavity is fluidly connected to the source of the pumped fluid through the inlet valve. The outlet port is disconnected from the pump cavity lower portion due the closure of the outlet valve.

At this time, the pressure supplied by the driving pressure source begins to decrease. As long as the driver pressure is higher than the fluid pressure at the inlet port, there will be no change in the state of the pump. When the pressure from the driver source is equal to the pressure at the inlet port, the pumped fluid begins to flow through the open inlet valve. The fluid flowing through the inlet valve will force the cavity divider up. During movement of the cavity divider, the pressure on both sides of the cavity divider will remain the same. At this time, the fluid pressure at the inlet port of the pump can be measured by fluidly connecting a pressure sensor to the tube connecting the driver pressure source to the upper portion of the pump cavity.

As the cavity divider reaches the top wall of the pump cavity, the internal volume of the lower portion of the cavity is now completely full and will be equal to the total usable volume of the pump cavity. The driver pressure source now continues to decrease the driver pressure until it reaches a second pressure level which causes the main piston to move from the down position to the up position, forcing both inlet and outlet valves to a new state in which the inlet port is disconnected from the cavity lower portion, while the cavity lower portion is fluidly connected through the outlet valve to the outlet port. The pumped fluid does not leave the cavity since there is no pressure present to force it out of the lower portion of the cavity.

At this time, the pressure source begins to increase the driver pressure, until this pressure becomes larger than the fluid pressure at the outlet port. This forces the cavity divider down, pushing the pumped fluid from the lower portion of the pump cavity, through the outlet valve to the outlet port. At this time, the fluid pressure at the outlet port of the pump can be measured by fluidly connecting a pressure sensor to the tube connecting the driver pressure source to the upper portion of the pump cavity.

The pressure from the driver pressure source continues to increase until it reaches the second pressure level, which causes the main piston to move from the up position to the down position, forcing the outlet valve to close and the inlet valve to open.

The next pumping cycle can begin when the main piston is in a down stable position, the cavity divider is in the second position, the inlet valve is open, the outlet valve is closed, and the driver pressure is positive and higher than the inlet pressure.

It can be beneficial to include at least one mechanical part to ensure that the main piston has only the two stable positions, the up position and the down position, and that the main piston will be unstable in any other position between the two. The mechanical part can be a loaded spring, a ball and groove setup, an electrical switch, magnets, or any other means which is well known in the art.

It can be beneficial to make the top and bottom surfaces of the cavity, against which the cavity divider is pressed in the first and second positions, as a network of shallow grooves rather than as flat surfaces with holes leading to the fluid paths to the ports. The grooves can ensure that the forces applied to the cavity divider at the maximum positive and negative pressures are evenly distributed, and can prevent breaking of a diaphragm membrane if the diaphragm is exposed to a too-high pressure at one point.

The cross-section of the inlet and outlet ports is preferably oval or slot shaped, with the top-bottom dimension much smaller than the left-right dimension. This allows the travel needed by the main piston to open and seal the openings to be shorter, and therefore reduces the depth of the pump.

The inlet port and outlet port comprise a pair of holes, with the pair of holes comprising a hole in the main piston matched to a hole in the side wall of the housing. The matched pair of holes can have a circular cross-section, or they can have a dimension along the axis of motion of the main piston which is in a range between 1.3- and 5 times smaller than the dimension along the axis that is perpendicular to the axis of motion of the main piston.

The tube connecting the control pressure inlet and the driver pump should preferably have a small diameter lumen in order to minimize the added volume and have non-stretchable walls in order to minimize changes in this volume due to internal pressure changes.

Note that, in this embodiment, the valves are opened and closed using the sliding motion of the main piston. The two holes, one in the main piston and one in the wall of the housing, either align, or not, depending on the position of the main piston. When the two holes are aligned, the valve is open, if they are not aligned, the valve is closed. This type of valve has the advantage of maintaining a good seal even when the fluid pumped may have particles or soft floaters which can interfere with the seal of a standard leaf valve. It can be beneficial to make the sliding surfaces of both the main piston and the housing wall very hydrophobic if pumping water-based fluids, to lower chances of leaks.

A novel feature of the present invention is the use of a highly flexible diaphragm (membrane) or a freely moving piston which conform to the contours of both the top and the bottom inner surfaces of the pump cavity when at rest in either the first or second positions. This feature assures that the pump will deliver a constant stroke volume of the pumped fluid. The elasticity, flexibility and compressibility of the diaphragm material(s) are selected and the cavity walls are manufactured so that the usable cavity volume is calibrated to provide the desired stroke volume.

A second novel feature of the invented pump is the use of active, pressure-actuated inlet and outlet valves, driven by the same pressure line that drives the pumping action, thus eliminating any valve related pumped fluid volume variations or errors, and simplifying pump construction.

A third novel feature is the use of active, pressure actuated inlet and outlet valves with activation pressures for switching from the up position to the down positions, and back again under the force of the driver pressure.

A fourth novel feature of the present invention is the ability to drive the pump and to measure pressure at both the inlet port and the outlet port through the single tube connecting the pump housing and the upper part of the cavity volume to the driver pressure source.

A fifth novel feature of the present invention is the ability to control the release of pumped fluid volumes which are smaller than the useful volume of the pump internal cavity, by controlling the volume of the fluid sent from the driver pressure source to the pump housing and the upper portion of the cavity at the time of equilibrium of pressures between the upper and lower portion of the cavity. This is manifested by the ability to pump additional volume of driving fluid without changing the pressure in the closed volume comprising the driver pressure source, volume of the tube connecting the driver source with the pump, and the upper portion of the pump volume.

A sixth novel feature is the ability to detect leaks of the driver fluid, as indicated by slow changes in the pressure in the up state or the down state, where there should be no change in volume, and therefore pressure, over time.

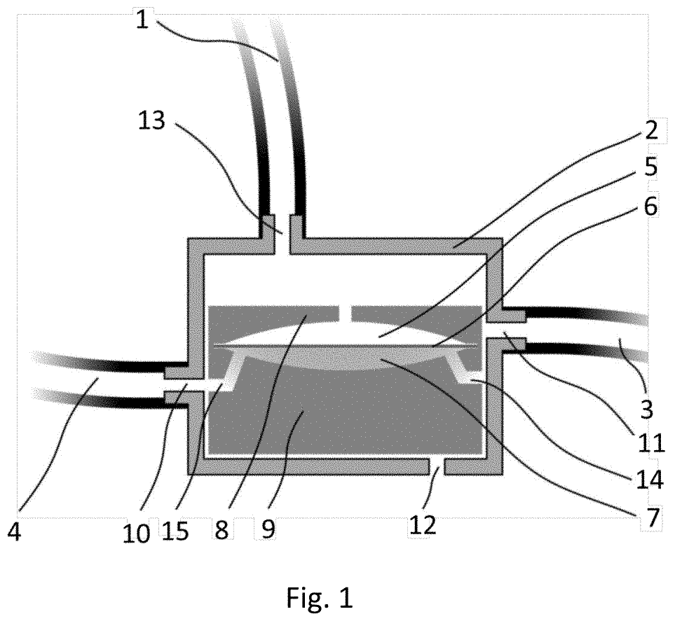

With reference to FIG. 1, in the embodiment, the pump comprises a pump housing 2 preferably manufactured from a hard thermoplastic material with a low coefficient of friction such as polyethylene (PE) or high density polyethylene (HDPE), having an internal volume and four fluid ports. The four ports can be integral parts of the housing and molded together with the housing in the same production step, or can be added at a later step using a plastic bonding technique. The inlet port 10 can be placed in fluid communication through a tube 4 with a source of pumped fluid. The outlet port 11 can be placed in fluid communication through a tube 3 with a reservoir or a drain that receives the pumped fluid. The control pressure source can be in fluid communication through a tube 1 with the pump housing via control port 13. The pressure sensing component(s) are in fluid communication with the pump housing, preferably via control port 13. The pressure sensing component(s) can be in fluid communication directly with the interior of the housing, via a mount on control port 13, or via tube 1. Venting port 12 is in fluid communication with ambient air, and is intended to release excess pressure which may be present under the main piston as it moves up and down.

In the housing internal volume, a main piston comprising a top part 8 and a bottom part 9 is movable towards the control port 13 and away from the control port 13; up and down in the embodiment shown. The main piston parts can be manufactured from low surface friction coefficient thermoplastic material, such as Teflon®. The two main piston parts 8 and 9 are joined together to form a movable main piston with an internal cavity having a very precise usable internal volume (consisting of volume 5 and volume 7, omitting the volume of diaphragm 6), and a very flexible diaphragm 6 dividing the cavity 5+7 into a first (upper) portion 5 and second (lower) portion 6. The diaphragm sealingly links the top and bottom main piston parts, preventing contact between fluid present in the upper portion of the cavity 5 and fluid present in the lower portion of the cavity 6.

The main piston thus has two stable positions, a stable up position in which its top surface coincides with the top surface of the housing internal space, with minimal clearance to ensure an even distribution of pressure across the main piston's entire top face, and a stable down position in which the main piston's bottom surface coincides with the bottom surface of the housing internal space.

The side faces of the main piston can slide over the internal side surfaces of the housing internal volume in such a way as to form tight-fitting contacting surfaces, which form a seal above and below each section or port level, including the entire main piston, thereby ensuring that the space above the main piston is fluidly isolated from the space below the main piston.

The lower portion of the piston cavity 7 can be in fluid communication with inlet tube 4 and inlet port 10 via fluid path 15 or can be in fluid communication with outlet tube 3 and outlet port 11 via fluid path 14, or there can be a single, joint fluid path shared by the inlet and outlet flow, made in the bottom part of the main piston 9 reaching the side surfaces of the main piston at two different, and vertically-separated locations designated the first 10 and second 11 openings (inlet and outlet ports). The relative locations of these openings are designed in such a way as to align first opening 15 with the location of the opening of the inlet port 10 when the main piston is at its bottom stable position, and to align the location of the second opening 14 with the location of the opening of the outlet port 11 when the main piston is at its top stable position.

It is thus clear that when the main piston 8+9 is at its bottom stable location, the bottom portion of the cavity 7 in the main piston 8+9 is in fluid communication with the inlet port 10 of the housing via the first opening, with the outlet port 11 sealed by the side surface of the main piston 8+9. On the other hand, when the main piston 8+9 is in its top stable position, the bottom portion of the cavity 7 in the main piston 8+9 is in fluid communication with the outlet port 11 of the housing via the second opening, with the inlet port 10 sealed by the side surface of the main piston 8+9. The air trapped under the main piston 8+9 as it moves up and down is allowed to escape to ambient through the vent port 12.

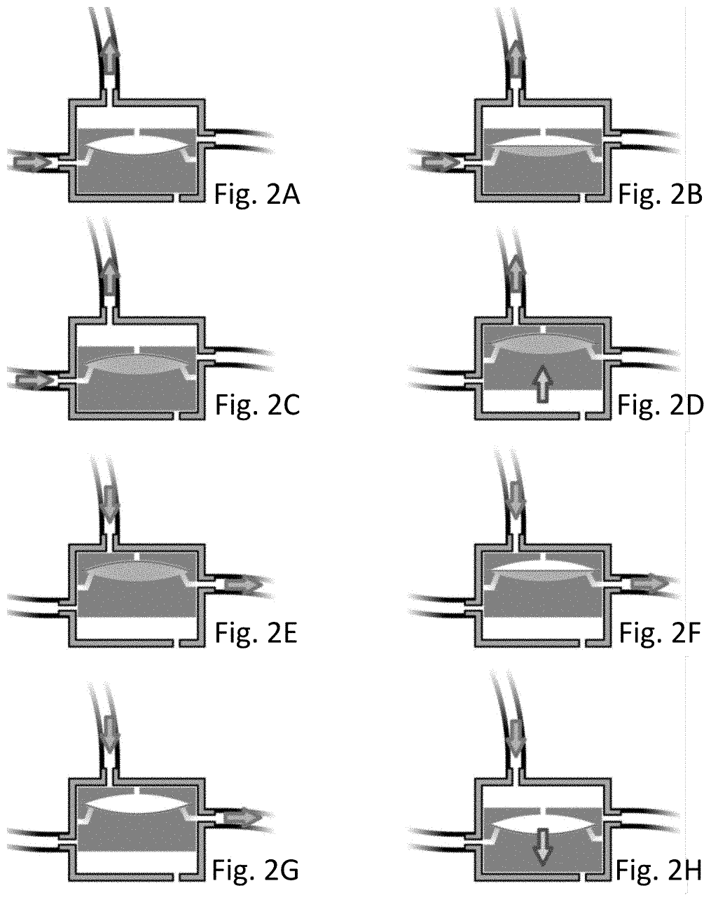

The pumping action of the pump can be better explained by looking at FIG. 2A-H.

In FIG. 2A, the main piston is in its down position, the diaphragm in the cavity is in the lower second position, and the lower part of the cavity is in fluid connection with the inlet port (on the left) connected to the source of pumped fluid. At this time, the driver pressure supplied from the driver port at the top of the housing is higher than the pressure of the fluid at the inlet port, so no fluid flows in through the port.

At this time, the driver pressure source starts lowering the driver pressure. Nothing happens until the driver pressure becomes equal to the inlet pressure, at which time the diaphragm in the cavity starts moving up from its second position to its first position as is shown in FIG. 2B. At this time, it is possible to measure the pressure in the inlet port by measuring the pressure in the driver port since they must be equal as long as the diaphragm has not reached the end of its travel.

The driver continues to pull fluid through the driver port (suction on the interior of the housing), and the diaphragm moves all the way up to the first position. After diaphragm has moved to the first position, the pressure above the main piston continues to decrease, as shown in FIG. 2C. The pressure above the main piston decreases until it is below atmospheric pressure. During this phase, the main piston moves up, as shown in FIG. 2D. The movement of the main piston closes the valve connecting the lower part of the cavity with the inlet port, and opens the valve connecting the lower part of the cavity with the outlet port. The fluid in the lower part of the cavity will not flow out because there is no pressure to induce the fluid to flow.

Once the pressure is low enough that the main piston is in the up position, the driver pressure source stops lowering the pressure above the main piston, and starts increasing it again, as shown in FIG. 2E. When the pressure in the driver port becomes equal to the pressure at the outlet port, the diaphragm will start moving down, pushing the fluid in the lower part of the cavity out through the outlet port, as shown in FIG. 2F. At this time, it is possible to measure the pressure in the outlet port by measuring the pressure in the driver port since they must be equal as long as the diaphragm has not reached the end of its travel.

The pressure in the driver port continues to increase until the diaphragm has moved all the way down and the lower part of the cavity is empty, as shown in FIG. 2G. The pressure continues to increase until the main piston moves down into its down position, as shown in FIG. 2H—returning the system to its initial state, ready for the next cycle.

In some embodiments, the driver fluid is air; fluids such as, but not limited to, water, oil or hydraulic fluid can be used as the driver fluid.

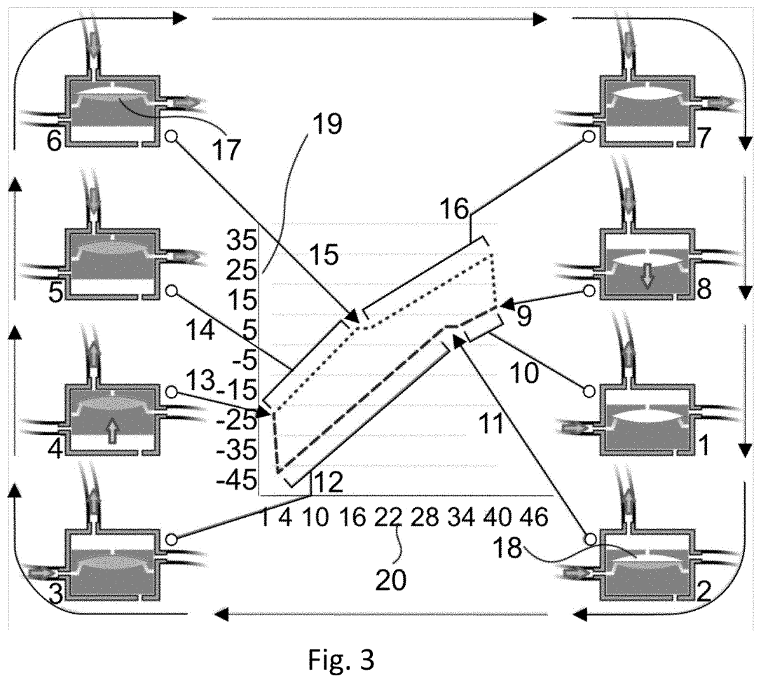

The pressure/volume loop of the driver source can be seen in FIG. 3. The X Axis 20 represents the change in volume of driver fluid caused by the driver pump, and the Y axis 19 represents a driver pressure difference, not the actual driver pressure.

Starting from the bottom right, pump state 1 represents the state right after the main piston has moved down, with maximum pressure at the driver port. The pressure in the volume above the main piston (inclusive of the volume of the cavity above the diaphragm) then decreases as the driver applies suction to the driver fluid, removing a predetermined volume of driver fluid. The slope of the graph 10 shows the pressure decreasing until, at pump state 2, the driver pressure becomes equal to the pressure at the inlet port and pumped fluid starts flowing into the cavity. As long as the diaphragm is moving 18, the pressure doesn't change even as more fluid is pumped out of the space above the main piston, as shown by graph section 11. When the diaphragm reaches the top of the cavity, and the cavity is therefore totally full, the pressure above the diaphragm starts dropping as shown in pump state 3, but now the space being evacuated is smaller than the space in section 10 since it does not include the usable volume of the cavity. The slope of the graph is therefore sharper.

The pressure continues to drop until, as shown in pump state 4, the main piston moves up. This creates a sudden drop in the pressure above the main piston, which signals the pressure drive to reverse direction. At pump state 5, the pressure above the main piston, as represented by graph section 14, increases until it reaches the pressure at the outlet port—state 6. The diaphragm 17 starts moving down, holding the pressure constant as long as it is moving, as shown by graph section 15. Once the diaphragm has moved all the way down, pressure continues to increase, as depicted by graph section 16 and pump state 7, after which the increased pressure forces the main piston down to close the loop as pump state 8, reaching graph state 9.

It is important to note that the slope of the graph when the diaphragm is in its down position differs from the slope of the graph when the diaphragm is in its up position, since the volume the drive pressure is acting on is different—the volume the drive pressure acts on becomes smaller or larger by the usable volume of the cavity. The total volume comprises the volume above the diaphragm in the cavity, the space above the main piston, the volume of the lumen of the tube connecting the control pump and the pump housing, and the volume of the control pump itself. Therefore, the rate of pressure change is different for points in the cycle where this total volume is different; the slope of the pressure vs. time graph is not the same when the diaphragm (or other cavity divider) is in the down position as when it is in the up position. This change in slope is important since it allows the system to find the location of the flat part of equal pressures (15 and 11) (when inlet and outlet pressures must be measured) even if it is too small to be otherwise detectable.

Since the cavity in the main piston, which defines the volume of fluid pumped in each cycle, may have slightly different volumes in different pumps due to production tolerances, or because residue can accumulate in the cavity or on the cavity divider, it can be useful to measure the active volume of the pump. This can be accomplished by measuring the change in volume of driver fluid induced by the driver pump, which does not lead to a change in pressure in the driver fluid line and in the space above the moving part. Since, at this time, the cavity divider is moving from being totally on one side of the cavity to being totally on the other side of the cavity, the change in volume of the driver fluid is exactly equal to the active volume of the cavity. Active volume means the volume of the cavity, minus the volume of any residues or dirt buildup on the cavity divider on the side facing the pumped fluid, which lowers that maximal volume of pumped fluid pumped in each cycle.

Since the volume of the cavity is small, the sections of the pressure loop where the pressure doesn't change and which represent the pressures at the inlet and outlet ports may be small and can be easily missed.

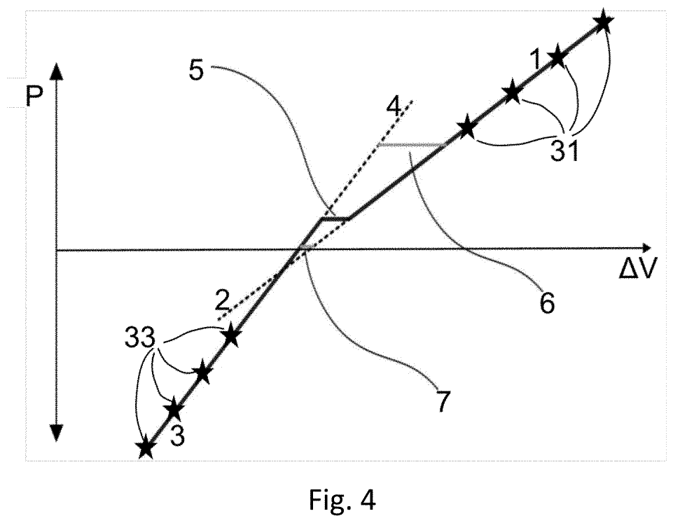

The location of these sections can be deduced from the change in slope of the volume/pressure loop measured by the pressure driver. This can be better explained in FIG. 4.

By measuring several data points (black stars 31 and 33) on both sides of the expected location of the flat (constant pressure) section, it is possible to apply linear regression to determine the equations of lines 1 and 3 as P=aV+b and P=cV+d respectively. Since the usable volume of the cavity, V_{c}, is known, it is possible to calculate the pressure during cavity divider movement, the inlet (or outlet) pressure P from aV_{c}+b=cV_{c}+d. The calculation is depicted by line 5, which has a length V_{c} while constant-pressure lines at different pressures 6 and 7 do not.

If perfect isolation between the pumped fluid and the fluid used to control and power the pump is needed, an elastic or collapsible tube such as a bellows can be added between the inside surface of the top of the housing and the top surface of the main piston. This way, even if some pumped fluid escapes the valves and wets the wall of the housing, it can't contaminate the control fluid circuit. This is especially important in medical applications, where the pumped fluid may present a biological hazard and must not contaminate the driving pump.

It should be noted that, in some embodiments, the cavity divider is not perpendicular to the piston axis of motion.

Since the pump is always an obstruction to the flow of the pumped fluid, some applications may warrant the addition of a fail-safe apparatus that will prevent the accumulation of too high a pressure in the inlet port if the pump stops working for any reason. One possible embodiment of such a safety pressure-release valve can be presented as making the surfaces comprising one half of each valve moveable in the direction perpendicular to the direction of motion. These can be designed as spring-loaded against their mating surfaces. If the pressure at any port is higher than the holding force of the springs, the fluid pressure will push the movable surfaces to form cracks in the seal through which fluid can escape the pump so as to release the extra pressure via venting port 12.

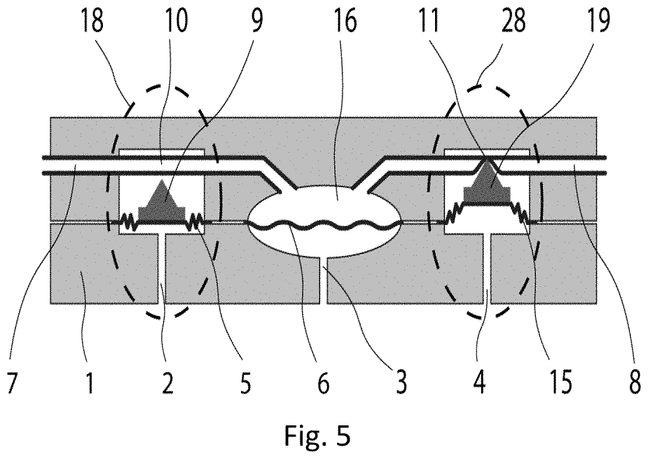

A second embodiment of the pump is shown in FIG. 5. In this embodiment, the pump body 1 comprises a cavity 16 divided by a diaphragm 6, a first fluid port 7 and a second fluid port 8. The cavity 16 is fluidly connected to a pressure source (not shown) via a pump cavity pressure inlet tube 3.

In this embodiment, active valves 19 and 21 (dashed ovals) independently control opening and closing of, respectively, the first fluid port 7 and the second fluid port 8. For illustrative purposes, in FIG. 5, inlet active valve 9 is shown open and outlet active valve 19 is shown closed.

Each active valve comprises a compressible tube (10 and 11) fluidly connecting the port (7 and 8) to the cavity 16. The compressible tube (10 and 11) can be pinched shut by an active valve pincher (9 and 19). In the embodiment shown, the valves are pneumatic valves. Pinch valves are typically pneumatic, hydraulic, motor-driven or solenoid operated. Any type of actively-driven, automatically-controllable valve which avoids contact between the flowable fluid and the valve mechanism and which avoids any contamination towards or from the environment is applicable.

In the pneumatic active valves 18 and 28, pressurized air can enter or leave via control pressure inlet tubes 2 and 4, respectively. The pressurized air moves active valve pinchers 9 and 19, respectively. Membranes 5 and 15 are sealingly connected to active valve pinchers 9 and 19, respectively, and sealingly connected to the exterior of the valve body, so that the control air is fluidly isolated from both the interior of the pump and from the pumpable fluid in the first fluid port 7, the second fluid port 8 and the cavity 16, thus preventing contamination of both the pumpable fluid and the control air and ensuring that all the control air pressure is used to pinch tubes 10 and 11, respectively.

In FIG. 5, the first active valve 18 is shown in an open position and the second active valve 28 is shown in a closed position. When the active valve is open (first active valve 18), the control air pressure is low and the active valve pincher 9 is retracted away from the compressible tube 10, allowing fluid to flow through compressible tube 10, and the membrane 5 is compressed.

The control air pressure is increased to close the active valve. When the active valve is closed (second active valve 28), the control air pressure is high and the active valve pincher 19 is extended, compressing the compressible tube 11, preventing fluid from flowing through compressible tube 11, and the membrane 15 is expanded.

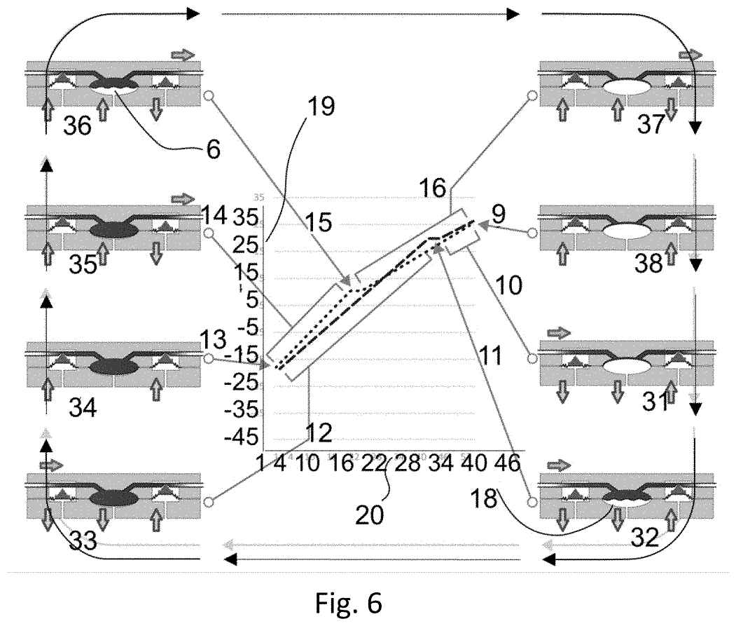

The pressure/volume loop of the driver source can be seen in FIG. 6. The X Axis 20 represents the change in volume of driver fluid caused by the driver pump, and the Y axis 19 represents a driver pressure difference, not the actual driver pressure. In FIG. 6, pumped fluid flows from left to right through the pump, as shown by the horizontal arrows.

A cycle of operation starts with the inlet valve 18 open, outlet valve 28 closed and the diaphragm 6 in its up position against a side of the cavity 16 opposite to the driver port 3. Suction is applied to the driver port 3, decreasing the pressure in the cavity 16 (31, 10 in FIG. 6). When the pressure in the cavity 16 has decreased until it is equal to the pressure at first fluid port 7, pumped fluid will flow through first fluid port 7, through valve 18 and into the cavity 16, forcing the diaphragm 6 downward and away from the wall of the cavity 16 against which it had been resting (32, 11 in FIG. 6). During this phase of the cycle, the pressure in the cavity 16 does not change, staying the same as the pressure at first fluid port 7. When the cavity 16 is full, with the diaphragm 6 resting against the side of the cavity 16 comprising the driver port 3, no more pumped fluid can flow into the cavity 16 and the pressure in the cavity 16 and at the driver port 3 will start to change (33, 12 in FIG. 6). Then, both valve 18 and valve 28 can be closed (34, 13 in FIG. 6). At this point, the pump will be put in a state with inlet valve 18 closed, and outlet valve 28 opened; positive pressure will be applied at the driver port 3, increasing the pressure in the cavity 16 (35, 14 in FIG. 6). When the pressure in the cavity 16 has increased until it is greater than the pressure at the second fluid port 8, the diaphragm 6 will be pushed away from the wall comprising the driver port 3, pushing the pumped fluid out through valve 28 and second fluid port 8 (36, 15 in FIG. 6). (After exit from second fluid port 8, it can be stored in a reservoir, let into a drain, be transferred for further processing, or otherwise treated in any conventional manner for fluid that is being pumped.) During this phase of operation, while the diaphragm 6 is moving, the pressure in the cavity 16 does not change (37, 16 in FIG. 6), staying the same as the pressure at second fluid port 8. When the cavity 16 is empty, with the diaphragm 6 resting against the side of the cavity 16 opposite to the driver port 3, cavity 16 is empty so that no more pumped fluid can flow out the cavity 16 and the pressure in the cavity 16 and at the driver port 3 will start to change. At this point, both valve 18 and valve 28 can be closed (38, 9 in FIG. 6), completing the cycle. When inlet valve 18 is opened and outlet valve 28 is closed, a new cycle starts.

As shown in FIG. 6, the pressure/volume loop of the driver source is similar to the pressure-volume loop of the driver source seen in FIG. 3. The first, suction, half of the cycle, 10 through 12, occurs when the inlet valve (18 in FIG. 5) is open and the outlet valve (28 in FIG. 5) is closed, while the second, pressure, half of the cycle, 14 through 16, occurs when the inlet valve (18 in FIG. 5) is closed and the outlet valve (28 in FIG. 5) is open.

Measurement of the pressure can be carried out in the same manner as for the first embodiment, with the at least one pressure sensor being in fluid communication with the driver fluid only. The at least one pressure sensor can be in fluid communication with any of the driver fluid side of the cavity 16 (the upper side of the cavity in the embodiment of FIG. 5), driver port 3, tubing attached to driver port 3, and the driver pressure source, but no pressure sensors are needed in fluid communication with the pumped fluid. There is no need to have a pressure sensor on any of first fluid port 7, second fluid port 8, valve 18, or valve 28, or in fluid communication with the pumped fluid side (the lower side in the embodiment of FIG. 5).

As in the first embodiment of the pump, the slope of the graph when the diaphragm 6 is in its down position differs from the slope of the graph when the diaphragm 6 is in its up position, since the volume the drive pressure is acting on is different—the volume the drive pressure acts on becomes smaller or larger by the usable volume of the cavity. The total volume comprises the volume above the diaphragm in the cavity, the volume of the lumen of the tube connecting the control pump and the pump housing, and the volume of the control pump itself. Therefore, the rate of pressure change is different for points in the cycle where this total volume is different; the slope of the pressure vs. time graph is not the same when the diaphragm 16 (or other cavity divider) is in the down position as when it is in the up position. This change in slope is important since it allows the system to find the location of the flat part of equal pressures (15 and 11) (when inlet and outlet pressures must be measured) even if it is too small to be otherwise detectable.

Since the cavity 16, which defines the volume of fluid pumped in each cycle, may have slightly different volumes in different pumps due to production tolerances, or because residue can accumulate in the cavity 16 or on the diaphragm 6, it can be useful to measure the active volume of the pump. This can be accomplished by measuring the change in volume of driver fluid induced by the driver pump which does not lead to a change in pressure in the driver fluid line. Since, at this time, the diaphragm 6 is moving from being totally on one side of the cavity 16 to being totally on the other side of the cavity 16, the change in volume of the driver fluid is exactly equal to the active volume of the cavity 16. Active volume means the volume of the cavity 16, minus the volume of any residues or dirt buildup on the diaphragm 16 on the side facing the pumped fluid, which lowers that maximal volume of pumped fluid pumped in each cycle.

Since the volume of the cavity 16 is small, the sections of the pressure loop where the pressure doesn't change and which represent the pressures at the inlet and outlet ports may be small and can be easily missed.

The location of these sections can be deduced from the change in slope of the volume/pressure loop measured by the pressure driver in the same manner as disclosed above in FIG. 4.

As shown in FIG. 4, by measuring several data points (black stars 31 and 33) on both sides of the expected location of the flat (constant pressure) section, it is possible to apply linear regression to determine the equations of lines 1 and 3 as P=aV+b and P=cV+d respectively. Since the usable volume of the cavity, V_{c}, is known, it is possible to calculate the pressure during cavity divider movement, the inlet (or outlet) pressure P from aV_{c}+b=cV_{c}+d. The calculation is depicted by line 5, which has a length V_{c} while constant-pressure lines at different pressures 6 and 7 do not.

If perfect isolation between the pumped fluid and the fluid used to control and power the pump is needed, an elastic or collapsible tube such as a bellows can be added between the inside surface of the top of the housing and the top surface of the main piston. This way, even if some pumped fluid escapes the valves and wets the wall of the housing, it can't contaminate the control fluid circuit. This is especially important in medical applications, where the pumped fluid may present a biological hazard and must not contaminate the driving pump.

It should be noted that, in some embodiments, the cavity divider is not perpendicular to the piston axis of motion.

In this embodiment of the pump, if pressure at the inlet port of the pump becomes too high, a fault indication can be raised and the system put into a free-flow state, where both valve 18 and valve 28 are open at the same time. In this free-flow state, the system adds little or no resistance to flow, allowing the excess pressure to be relieved.

In embodiments with independent, actively-controlled valves, since each valve is controlled independently, whether a given valve is open or closed is unaffected by either the state of other valves in the system or the state of the diaphragm in the cavity. This provides additional flexibility in the system. In such embodiments, it is possible to:
1. Pump in both directions by changing the time relationship between the diaphragm or other cavity divider and the valve activations.
2. Check that there are no leaks in the valves by closing both and trying to push fluid out. A leak can be detected by a drop in pressure in the pumping cavity.
3. Have no pressure drop across open valves, so that pressure can be measured independently at both the first and second ports.

It should be noted that, in some embodiments, there can be one or more first ports and that the plurality of first ports can be controlled by one or more valves. Similarly, in some embodiments, there can be one or more second ports and that the plurality of second ports can be controlled by one or more valves.

1-48. (canceled)

49. A positive displacement pump comprising:
a housing having at least two pumping ports for flowing a pumpable fluid into and out of the pump and at least one control port for flowing operating pressure into and out of the pump;
at least one cavity inside the housing, divided into a first volume and a second volume by a diaphragm, said first volume fluidly isolated from said second volume, said second volume fluidly connectable to said at least two pumping ports, said first volume fluidly connected to said at least one control port; said diaphragm movable by means of said operating pressure; said second volume reversibly enlargeable by movement of said diaphragm; and
at least two valves configured to control flow through said at least two pumping ports, fluid flow through all said at least two pumping ports controllable by at least one of said at least two valves, a first at least one of said at least two valves in fluid connection with a first at least one of said at least two pumping ports, a second at least one of said at least two valves in fluid connection with a second at least one of said at least two pumping ports, control of at least one of said at least two valves being independent of control of at least one other of said at least two valves;
wherein enlargement of said second volume flows said pumpable fluid into said pump and reversing enlargement of said second volume flows said pumpable fluid out of said pump;
further wherein said diaphragm is a flexible plastic film, with a thickness in a range of 0.01 mm-0.5 mm, allowing movement of said diaphragm up and down in said at least one cavity with minimal loss of pressure and leading to a movement of said diaphragm between a top of said at least one cavity and a bottom of said at least one cavity that does not affect a cavity pressure within said at least one cavity, a port pressure at at least two of said at least two pumping ports being thereby determinable from measurement of said operating pressure.

50. The pump of claim 49, wherein control of at least one of said at least two valves is independent of control of said operating pressure.

51. The pump of claim 49, additionally comprising at least one vent port in fluid communication with said second volume.

52. The pump of claim 49, wherein at least one of said at least two valves is a pinch valve.

53. The pump of claim 49, wherein said pinch valve is selected from a group consisting of a pneumatic-controlled valve, a hydraulic-controlled valve, a motor-driven valve, and a solenoid operated valve.

54. A positive displacement pump comprising:
a housing having at least three fluid ports, at least one inlet port configured to allow flow of a pumped fluid into the pump, at least one outlet port configured to allow flow of said pumped fluid out of the pump, and at least one driver pressure port configured to allow operating pressure into and out of the pump;
at least one main piston movable within a space defined by at least one first interior wall of the housing between a first stable position and a second stable position under said operating pressure, said operating pressure acting in a space between the main piston and at least one second interior wall of the housing;
at least one cavity coupled to or formed inside the main piston, said cavity divided into a first volume and a second volume by a freely moving divider;
at least one first valve, operable by motion of said main piston, configured to provide fluid connection between said at least one inlet port and said first volume when the main piston in said first stable position; and
at least one second valve, operable by the motion of said main piston, configured to provide fluid connection between said at least one outlet port and said first volume when the main piston in said second stable position;
wherein said pump is operable by a single pressure, said operating pressure, pumping of fluid between the inlet port and the outlet port being controlled by said operating pressure, and fluid pressure at said inlet port and said outlet port determinable from measurement of said operating pressure.

55. The pump of claim 54, wherein the divider separating the cavity into said first volume and said second volume is selected from a group consisting of a diaphragm and a second piston.

56. The pump of claim 55, wherein said diaphragm comprises a flexible plastic film with a thickness in a range of 0.01 mm-0.5 mm.

57. The pump of claim 54, wherein the pressure at the inlet port is determinable from the pressure at the driver pressure port, during a time in which a change of driver fluid volume in the space between the main piston and at least one second interior wall of the housing does not result in a pressure change in said space between the main piston and said at least one second interior wall of the housing, and the main piston in the said first position.

58. The pump of claim 54, wherein the pressure at the outlet port is determinable from the pressure at the driver pressure port, during a time in which a change of driver fluid volume in the space between the main piston and said at least one second interior wall of the housing does not result in a pressure change in said space between the main piston and said at least one second interior wall of the housing, and the main piston in the said second position.

59. The pump of claim 54, wherein the driver pump inverts its direction of operation after detection of a sudden drop in absolute pressure in the space between the main piston and said at least one second interior wall of the housing, said sudden drop on absolute pressure indicating that the main piston has moved from one stable position to the other stable position.

60. The pump of claim 54, wherein the pressure at the outlet or the inlet port is calculable by a process comprising the steps of:
measuring several data points on the curve representing the relationship between a change in volume of driver fluid and the pressure in the space between the main piston and said at least one second interior wall of the housing, said pressure being equal to pressure at the driver pump port;
calculating the parameters of two straight lines for the process of increasing the pressure from minimum value to maximum value, and two straight lines for the process of decreasing the pressure from the maximum value to the minimum value, having a formula as P₁=a₁(ΔV)+b₁ and P₂=a₂(ΔV)+b₂. One line is before the diaphragm moves from one stable position to another stable position, thereby either increasing or decreasing volume of the space between the main piston and said at least one second interior wall of the housing, and a second line after the diaphragm moves from one position to another position;
calculating the P₁=P₂ where a₁(ΔV)+b₁=a₂(ΔV)+b₂ where ΔV is equal to the volume of the cavity inside the main piston; and
from the pressure at the driver pressure port, when a change of driver fluid volume in the space between the main piston and said at least one second interior wall of the housing does not result in a pressure change in said space between the main piston and said at least one second interior wall of the housing, and the main piston is in the said second position.

61. The pump of claim 54, wherein a volume in the cavity available to the pumpable fluid is measurable as a change in volume of the driver fluid, said change in volume of the driver fluid not inducing a change in pressure in the space between the main piston and said at least one second interior wall of the housing.

62. The pump of claim 54, wherein the driver pump reverses its direction of operation from increasing the pressure to decreasing the pressure and vice versa, at such time as is measured a sudden drop in the absolute value of the pressure in the space between the main piston and said at least one second interior wall of the housing said sudden drop in pressure indicating that the main piston has moved from one stable position to the other stable position.

63. The pump of claim 54, wherein a leak in a fluid connection between a driver pressure pump and the driver pressure port is detectable from a continuation of the change in measured pressure towards zero pressure after the driver pressure pump, having a non-zero driver pressure, is stopped.

64. The pump of claim 54, wherein a malfunction condition of the main piston is detectable from a smaller-than-normal drop in absolute pressure when the main piston is moving at either maximal operating pressure or minimal operating pressure, said malfunction being failure of said main piston to move fully from one stable position to the other stable position.

65. The pump of claim 54, wherein at least one volume of pumped fluid smaller than a usable volume of the cavity is releasable by the pump, by operating the driver pressure pump when the operating pressure is equal to the outlet port pressure, and injecting a predetermined volume of driver fluid smaller than a usable volume of the cavity.

66. The pump of claim 54, wherein the diaphragm dividing the cavity lies in a plane that is not perpendicular to the axis of motion of the moving part.

67. The pump of claim 54, wherein each of said inlet port and said outlet port comprise a pair of holes, said pair of holes being a hole in said main piston matched to a hole in a portion of said at least one first wall of said housing, and said pair of holes have a dimension along an axis of motion of the main piston in a range between 1.3 and 5 times smaller than a dimension along an axis perpendicular to the axis of motion of the main piston.

68. A method of operating a positive displacement pump comprising steps of:
providing a positive displacement pump comprising:
a housing having at least two pumping ports for flowing a pumpable fluid into and out of the pump and at least one control port for flowing operating pressure into and out of the pump;
at least one cavity inside the housing, divided into a first volume and a second volume by a diaphragm, said first volume fluidly isolated from said second volume, said second volume fluidly connectable to said at least two pumping ports, said first volume fluidly connected to said at least one control port; said diaphragm movable by means of said operating pressure; said second volume reversibly enlargeable by movement of said diaphragm; and
at least two valves configured to control flow through said at least two pumping ports, fluid flow through all said at least two pumping ports controllable by at least one of said at least two valves, a first at least one of said at least two valves in fluid connection with a first at least one of said at least two pumping ports, a second at least one of said at least two valves in fluid connection with a second at least one of said at least two pumping ports, control of at least one of said at least two valves being independent of control of at least one other of said at least two valves;
connecting at least one of said at least two pumping ports to a source of fluid; and
executing at least one pump stroke, said pump stroke comprising:
opening said first at least one of said at least two valves and closing said second at least one of said at least two valves and decreasing said operating pressure, thereby enlarging said second volume and flowing said pumpable fluid into said second volume; and
at such time as said second volume is fully enlarged, closing said first at least one of said at least two valves and opening said second at least one of said at least two valves and increasing said operating pressure, thereby reversing enlargement of said second volume and flowing said pumpable fluid out of said pump
wherein said diaphragm is a flexible plastic film, with a thickness in a range of 0.01 mm-0.5 mm, allowing movement of said diaphragm up and down in said at least one cavity with minimal loss of pressure and leading to a movement of said diaphragm between a top of said at least one cavity and a bottom of said at least one cavity that does not affect a cavity pressure within said at least one cavity, a port pressure at at least two of said at at least two pumping ports being thereby determinable from measurement of said operating pressure.
